(19) United States
(12) Patent Application Publication
(10) Pub. No.: US 2026/0235623 A1
(43) Pub. Date: Aug. 13, 2026

(54) EFFECT INDUCED BY A SODIUM-GLUCOSE COTRANSPORTER INHIBITOR (SGLTi) IN A SUBJECT SUFFERING FROM A CARDIOVASCULAR DISEASE

(71) Applicant: Roche Diagnostics Operations, Inc., Indianapolis, IN (US)

(72) Inventors: Ying He, Penzberg (DE); Peter Kastner, Penzberg (DE); Anika Mang, Penzberg (DE); Serge Masson, Rotkreuz (CH); Andre Ziegler, Rotkreuz (CH)

(21) Appl. No.: 19/151,861

(22) PCT Filed: Jan. 30, 2024
(86) PCT No.: PCT/EP2024/052125

§ 371 Date: Jul. 29, 2025

(30) Foreign Application Priority Data
Jan. 30, 2023 (EP) 23153917.2

(51) Int. Cl. G01N 33/68

(52) U.S. Cl. CPC G01N 33/6893; G01N 2333/475; G01N 2333/58; G01N 2500/00; G01N 2800/32

(57) ABSTRACT

The present invention relates to a method of detecting the effect induced by a sodium-glucose cotransporter inhibitor (SGLTi) in a subject suffering from a cardiovascular disease, the method comprising a) determining the amount of Fatty Acid Binding Protein 3 (FABP3) in a sample of the subject treated with SGLTi; and b) comparing the amount of said FABP3 to a reference amount. Also encompassed are computer-implemented methods, computer program products, devices and kits for carrying out the method of the present invention. The present invention further relates to the use of FABP3, the use of FABP3 and a cardiac injury marker, and/or the use of a detection agent of FABP3 or FABP3 and a cardiac injury marker for detecting the effect induced by a sodium-glucose cotransporter inhibitor (SGLTi) in a subject suffering from a cardiovascular disease.

### FIELD OF THE INVENTION

The present invention relates to a method of detecting the effect induced by a sodium-glucose cotransporter inhibitor (SGLTi) in a subject suffering from a cardiovascular disease, the method comprising a) determining the amount of Fatty Acid Binding Protein 3 (FABP3) in a sample of the subject treated with SGLTi; and b) comparing the amount of said FABP3 to a reference amount. Also encompassed are computer-implemented methods, computer program products, devices and kits for carrying out the method of the present invention. The present invention further relates to the use of (i) FABP3, or FABP3 and a cardiac injury marker, (ii) a detection agent of FABP3, or FABP3 and a cardiac injury marker, for detecting the effect induced by a sodium-glucose cotransporter inhibitor (SGLTi) in a subject suffering from a cardiovascular disease.

### BACKGROUND OF THE INVENTION

Cardiovascular diseases severely affect the function of the heart and often result in the progression of heart failure, being the leading cause of morbidity and mortality in the Western hemisphere. Heart failure (HF) is a complex clinical syndrome that can result from any structural or functional cardiac disorder that impairs the ventricle's ability to fill with or eject blood and to safeguard the body's metabolic needs for supply with blood/oxygen. HF affects around 64 million of patients worldwide, and its prevalence is increasing due to population ageing, the growing burden of comorbidities and risk factors for HF, and the longer survival after myocardial infarction (see, e.g., Castiglione V, Aimo A, Vergaro G, Saccaro L, Passino C and Emdin M. Biomarkers for the diagnosis and management of heart failure. Heart Failure Reviews (2022) 27:625-643).

The therapeutic goals in heart failure are to improve survival, to prevent hospital-admissions and to relieve symptoms and signs of heart failure (HF), as e.g. congestion, dyspnea and fatigue (McMurray J J, Adamopoulos S, Anker S D, Auricchio A, Bohm M, Dickstein K, et al. ESC guidelines for the diagnosis and treatment of acute and chronic heart failure 2012: The Task Force for the Diagnosis and Treatment of Acute and Chronic Heart Failure 2012 of the European Society of Cardiology. Developed in collaboration with the Heart Failure Association (HFA) of the ESC. European journal of heart failure. 2012;14(8):803-69). Current standards of care for patients with heart failure include β-blockers, RAAS inhibitors, angiotensin receptor-neprilysin inhibitors and diuretics, to suppress neurohormones, reduce volume overload and thereby improve cardiac contractility (Ponikowski P, Voors A A, Anker S D, Bueno H, Cleland J G F, Coats A J S, et al. 2016 ESC Guidelines for the diagnosis and treatment of acute and chronic heart failure: The Task Force for the diagnosis and treatment of acute and chronic heart failure of the European Society of Cardiology (ESC) Developed with the special contribution of the Heart Failure Association (HFA) of the ESC. European heart journal. 2016;37(27):2129-200). Despite important cardiovascular benefits, a risk for acute decompensation, hospitalization and mortality remains and further treatment options are required. Hence, although available treatment options can reduce morbidity and mortality in patients with HF, the relative number of eligible patients receiving these treatments remains unsatisfactorily low (O'Donoghue M. & Braunwald E., Nat. Rev. Cardiol. 2010;7:13-20). Thus, an aim of modem medicine is to provide personalized or individualized treatment guidance which take into account a patient's individual needs or risks. However, especially for recently available treatment options, the mode of action may be incompletely resolved so that first of all a better understanding of such treatment options is needed.

Inhibitors of sodium-glucose cotransporters (SGLT), mainly inhibitors of Sodium-glucose cotransporter 2 inhibitors (SGLT2i) and Sodium-glucose cotransporter 1 inhibitors (SGLT1i), have attracted attention as therapeutic targets. Sodium-glucose cotransporters (SGLT) constitute a large family of membrane proteins with at least six different isoforms in humans and which mediate the transport of glucose across cellular membranes into cells, particularly across the apical membrane of the lumen side (see, e.g., Sabino-Silva R et al. The Na(+)/glucose cotransporters: from genes to therapy. Brazilian Journal of Medical and Biological Research 2010. 43 (11): 1019-26). SGLT inhibitors have been developed as an antidiabetic treatment by inhibiting glucose reabsorption and increasing excretion of glucose into urine (see, e.g., Ferrannini E. Sodium-glucose co-transporters and their inhibition: clinical physiology. Cell Metab 2017;26:27-38). Further, SGLT2i was found to confer protection against cardiovascular outcomes leading to changes in clinical recommendations and practice (see, e.g., Professional Practice Committee. Standards of medical care in diabetes-2019. Diabetes Care 2019;42(Suppl. 1): S3). The mechanism of action by SGLTi in patients having a cardiovascular disease is not fully understood. Accordingly, a better understanding of SGLTi treatment in patients suffering from a cardiovascular disease is needed. Having a marker that would allow the assessment/visualization/detection of the effect(s) induced by a sodium-glucose cotransporter inhibitor (SGLTi) in a subject suffering from a cardiovascular disease could also help to increase said understanding.

Therefore, the technical problem underlying the present invention can be seen as the provision of means and methods for complying with the aforementioned needs. The technical problem is solved by the embodiments characterized in the claims and herein below.

### SUMMARY OF THE INVENTION

The present invention provides a method of detecting the effect induced by a sodium-glucose cotransporter inhibitor (SGLTi) in a subject suffering from a cardiovascular disease, the method comprising a) determining the amount of Fatty Acid Binding Protein 3 (FABP3) in a sample of the subject treated with SGLTi; b) comparing the amount of said FABP3 to a reference amount. Further, the present invention provides a computer-implemented method for carrying out the method of the present invention, comprising a) means for determining the amount of FABP3, or the amount of FABP3 and a cardiac injury marker, in a sample of a subject treated with SGLTi, wherein the subject suffers from a cardiovascular disease; and b) means for comparing said amount of FABP3 to a reference amount, said amount of FABP3 and said amount of the cardiac injury marker to a reference, or means for forming and comparing a ratio between said amount of FABP3 and said amount of the cardiac injury marker to a reference ratio.

The present invention also provides a computer program product comprising a program code for executing the computer-implemented method of the present invention when run on at least one computer.

The present invention also provides a device for carrying out the method of the present invention, comprising a) means for determining the amount of FABP3, or the amount of FABP3 and a cardiac injury marker, in a sample of a subject treated with SGLTi, wherein the subject suffers from a cardiovascular disease; and b) means for comparing said amount of FABP3 to a reference amount, said amount of FABP3 and said amount of a cardiac injury marker to a reference amount, or means for forming and comparing a ratio between said amount of FABP3 and said amount of the cardiac injury marker to a reference ratio.

The present invention also provides a kit for carrying out the method of the present invention, comprising a) means for determining the amount of FABP3, or the amount of FABP3 and a cardiac marker, in a sample of a subject treated with SGLTi, wherein the subject suffers from a cardiovascular disease; and b) means for comparing said amount of FABP3 to a reference amount, said amount of FABP3 and said amount of the cardiac marker to a reference amount, or means for forming and comparing a ratio between said amount of FABP3 and said amount of the cardiac marker to a reference ratio.

The present invention also provides the use of i) FABP3, the use of FABP3 and a cardiac injury marker, and/or (ii) a detection agent of FABP3 or FABP3 and a cardiac injury marker, for detecting the effect induced by a sodium-glucose cotransporter inhibitor (SGLTi) in a subject suffering from a cardiovascular disease.

### BRIEF DESCRIPTION OF THE DRAWINGS

The invention will be better understood with reference to the detailed description when considered in conjunction with the non-limiting examples and the drawings, in which:

FIG. 1: Initiation of SGLT2i therapy is associated with an increase in plasma concentration of FABP3. FABP3 concentration expressed in ng/ml; boxplot showing median concentration, lower and upper quartile boundaries. V0: baseline (median FABP3=37.82), V1: week 4 after SGLT2i therapy initiation (median FABP3=40.86), V2; 3 months (median FABP3=45.14), V3: 6 months (median FABP3=44.36), V4: 12 months (FABP3=44.47).

FIG. 2: Changes over time after SGLT2i therapy initiation of FABP3 concentration in patients with high baseline FABP3 concentration (above the median concentration of 37.8 ng/ml). FABP3 concentration expressed in ng/ml; boxplot showing median concentration, lower and upper quartile boundaries. V0: baseline (median FABP3=47.97), V1: week 4 after SGLT2i therapy initiation (median FABP3=53.56), V2; 3 months (median FABP3=54.73), V3: 6 months (median FABP3=54.51), V4: 12 months (FABP3=53.23).

FIG. 3: Changes over time after SGLT2i therapy initiation of FABP3 concentration in patients with low baseline FABP3 concentration (below the median concentration of 37.8 ng/mL). FABP3 concentration expressed in ng/ml; boxplot showing median concentration, lower and upper quartile boundaries. V0: baseline (median FABP3=29.05), V1: week 4 after SGLT2i therapy initiation (median FABP3=31.41), V2; 3 months (median FABP3=38.86), V3: 6 months (median FABP3=36.41), V4: 12 months (FABP3=36.76).

FIG. 4: Changes over time after SGLT2i therapy initiation of the ratio of FABP3/NT-proBNP concentration. FABP3 concentration expressed in ng/ml, NT-proBNP concentrations expressed in pg/mL; boxplot showing median ratio of FABP3/NT-proBNP, lower and upper quartile boundaries. V0: baseline (median FABP3/NT-proBNP ratio=0.026), V1: week 4 after SGLT2i therapy initiation (median FABP3/NT-proBNP ratio=0.032), V2; 3 months (median FABP3/NT-proBNP ratio=0.039), V3: 6 months (median FABP3/NT-proBNP ratio=0.046), V4: 12 months (median FABP3/NT-proBNP ratio=0.033).

FIG. 5: Changes over time after SGLT2i initiation of circulating NT-proBNP concentration. NT-proBNP concentration expressed in pg/mL; boxplot showing median concentration, lower and upper quartile boundaries. V0: baseline (median NT-proBNP=1396.30), V1: week 4 after SGLT2i therapy initiation (median NT-proBNP=1115.68), V2: 3 months (median NT-proBNP=1191.95), V3: 6 months (median NT-proBNP=889.21), V4: 12 months (median NT-proBNP=1172.84).

FIG. 6: Changes over time after SGLT2i initiation of the ratio of circulating FABP3/GDF-15 concentration. FABP3 concentration expressed in ng/ml, GDF-15 concentrations expressed in pg/mL; boxplot showing median ratio of FABP3/GDF-15, lower and upper quartile boundaries. V0: baseline (median FABP3/GDF-15 ratio=0.017), V1: week 4 after SGLT2i therapy initiation (median FABP3/GDF-15 ratio=0.016), V2; 3 months (median FABP3/GDF-15 ratio=0.016), V3: 6 months (median FABP3/GDF-15 ratio=0.016), V4: 12 months (median FABP3/GDF-15 ratio=0.019).

FIG. 7: Changes over time after SGLT2i initiation of circulating GDF-15 concentration. GDF-15 concentration expressed in pg/mL; boxplot showing median concentration, lower and upper quartile boundaries. V0: baseline (median GDF-15=2439.34), V1: week 4 after SGLT2i therapy initiation (median GDF-15=2657.16), V2; 3 months (median GDF-15=3036.18), V3: 6 months (median GDF-15=2414.01), V4: 12 months (median GDF-15=2039.85).

### DETAILED DESCRIPTION

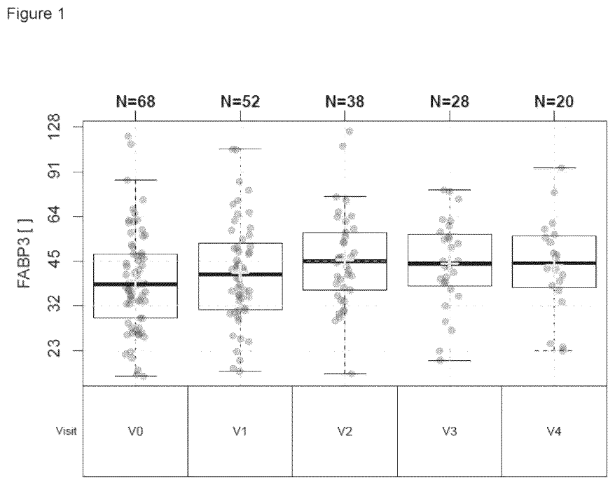

The present invention relates to detecting the effect induced by a sodium-glucose cotransporter inhibitor (SGLTi) in a subject having a cardiovascular disease, the method comprising (a) determining the amount of Fatty Acid Binding Protein 3 (FABP3) in a sample of the subject treated with SGLTi; (b) comparing the amount of said FABP3 to a reference amount. Accordingly, the present invention is based on the discovery that the effect induced by a SGLTi in a subject suffering from a cardiovascular disease correlates with the amount of FABP3. Particularly, as demonstrated in the experimental section herein, large scale analysis of samples reveals that in patients having heart failure, treatment with SGLT2i correlates with the amount of FABP3 in said patients; see FIG. 1. FABP3 can therefore be used to detect/visualize/assess the effect induced by a SGLTi in a subject suffering from a cardiovascular disease. Particularly, as demonstrated in the experimental section herein, concentration of the amount of FABP3 increases with the treatment of SGLTi (and thereby visualizes or assess the SGLTi treatment), wherein the increase is apparent for at least up to 12 months. Therefore, the effect induced by SGLTi can reliably be detected (visualized). “Detection” thus may be seen as “visualizing” or “assessing”. Further, the method of the invention allows for monitoring the effect induced by SGLTi in a subject suffering from a cardiovascular disease by (a) determining the amount of Fatty Acid Binding Protein 3 (FABP3) in a sample of the subject treated with SGLTi; (b) comparing the amount of said FABP3 to a reference amount. Monitoring and/or detecting (such as visualising or assessing) the effect induced by SGLTi based on the amount of FABP3 can, for example, be used to develop individualized treatment regimens such as for guiding, adjusting or changing a therapy with SGLTi in a patient suffering from a cardiovascular disease and thus can support physicians in making clinical decisions regarding the therapy with SGLTi.

Sodium-glucose cotransporter (SGLT) are located in the renal proximal tubular epithelium and mainly function by reabsorbing filtered glucose (see, e.g., Bakris GL, Fonseca VA, Sharma K, Wright EM. Renal sodium-glucose transport: role in diabetes mellitus and potential clinical implications. Kidney international. 2009;75(12):1272-7; Ferrannini E, Murthy A C, Lee Y, Muscelli E, Weiss S, Ostroff R M, Sattar N, Williams SA and Ganz P. Mechanisms of Sodium-Glucose Cotransporter 2 Inhibition: Insights From Large-Scale Proteomics. Diabetes Care (2020); 43:2183-2189). Inhibitors of SGLT have been reported to result in urinary excretion of glucose, sodium and water, leading to hemodynamic changes as well as glycemic improvements in patients with type 2 diabetes. Further, SGLT2i have been reported to decrease the risk of cardiovascular events. Despite the effect on diuresis, it is suggested that SGLT-inhibitors might have further cardioprotective local and systemic mechanisms of action that are not well understood until now. Without wishing to be bound by theory, one explanation for the beneficial therapeutic effect of SGLT2i in cardiovascular disease such as heart failure may be due to the primary action of SGLT2i on the proximal renal tubule inducing a range of physiological consequences in several domains of bodily functions (see, e.g., Ferrannini E, Murthy AC, Lee Y, Muscelli E, Weiss S, Ostroff RM, Sattar N, Williams SA and Ganz P. Mechanisms of Sodium-Glucose Cotransporter 2 Inhibition: Insights From Large-Scale Proteomics. Diabetes Care (2020); 43:2183-2189). Further, it is reported that SGLT2i may induce natriuresis and osmotic diuresis with blood volume contraction and increased hematocrit which may be responsible for the reduction in blood pressure and arterial stiffness, which can improve cardiac function by decreasing both pre-and afterload (see, e.g., Verma S, McMurray JJV. SGLT2 inhibitors and mechanisms of cardiovascular benefit: a state of-the-art review. Diabetologia 2018; 61:2108-2117). Concluding this review, the mechanisms by which SGLT2i confers its effect in a subject suffering from a cardiovascular disease are not completely understood. SGLTi is reported to induce a fasting-like metabolic paradigm involving the metabolic switch from carbohydrates to lipid utilisation and ketogenesis, which activates nutrient deprivation pathways and contributes to maintaining energy homeostasis and cardiorenal protection (see e.g., Gao Y M, Feng S T, Wen Y, Tang T, Wang B, Liu B C. Cardiorenal protection of SGLT2 inhibitors-Perspectives from metabolic reprogramming. EBioMedicine. 2022; 83:104215. doi: 10.1016/j.ebiom.2022.104215), such as evidence on changes in FABP3 levels in diabetic, non HF patients (Ferrannini E, Murthy AC, Lee Y, Muscelli E, Weiss S, Ostroff RM, Sattar N, Williams SA and Ganz P. Mechanisms of Sodium-Glucose Cotransporter 2 Inhibition: Insights From Large-Scale Proteomics. Diabetes Care (2020); 43:2183-2189). Thus, without wishing to be bound by theory, since the function of FABP3 is reported to comprise the transport of free fatty acids (see e.g., Storch et al., Biochem. Biophys. Acta. 1486(2000), 28-44), the correlation of FABP3 with the effect induced by SGLTi can be postulated to result from a metabolic switch from carbohydrates to lipid utilisation and ketogenesis by SGLTi.

Other markers which are commonly known by the skilled person to be implicated with cardiovascular disease, particularly heart failure, failed to detect the effect induced by SGLTi. For example, growth differentiation factor-15 (GDF-15) is expressed by numerous cell types, including cardiomyocytes, smooth and endothelial muscle cells, and the increase in plasma GDF-15 is reported to have a prognostic role in heart failure and was also found to be useful to identify a patient having heart failure to respond to a therapy comprising statin (see, e.g., Castiglione V, Aimo A, Vergaro G, Saccaro L, Passino C and Emdin M. Biomarkers for the diagnosis and management of heart failure. Heart Failure Reviews (2022) 27:625-643). However, as demonstrated in the experimental section herein, the amount of GDF-15 does not essentially change in subjects treated with SGLT2i medication. Further, the role of B-type NP (BNP) and N-terminal pro-B-type NP (NT-proBNP) for diagnosis and risk stratification in heart failure has been reported (see, e.g., Castiglione V, Aimo A, Vergaro G, Saccaro L, Passino C and Emdin M. Biomarkers for the diagnosis and management of heart failure. Heart Failure Reviews (2022) 27:625-643). However, as also demonstrated in the experimental section herein, NT-proBNP alone does not significantly change with the treatment of SGLTi, but FABP3 used in combination with NT-proBNP as demonstrated in the experimental section can be reliably used for detecting the effect induced by SGLTi, wherein a ratio between the amount of FABP3 and the amount of NT-proBNP correlates with the effect induced by SGLTi. Thus, as shown in the experimental section herein, markers known to be implicated with a cardiovascular disease such as GDF-15 or NT-proBNP alone, do not indicate the effect induced by SGLTi in a subject suffering from a cardiovascular disease. Instead, it was surprising that the effect is detectable via the amount of FABP3 or the amount of FABP3 in combination with NT-proBNP. Fatty-acid-binding proteins (FABPs) belong to a family of carrier proteins for fatty acids and other lipophilic substances. They are both membrane-bound-aiding cellular long-chain fatty acid uptake- and cytoplasmic, being crucial to intracellular transport of fatty acids to site of metabolic conversion. Hence, FABPs are ubiquitous and the 9 family members of FABPs are distributed in a variety of tissues. The FABP family includes liver (L-FABP/FABP1), intestinal (I-FABP/FABP2), heart (H-FABP/FABP3), adipocyte (A-FABP/FABP4/aP2), epidermal (E-FABP/FABP5/mal1), ileal (II-FABP/FABP6), brain (B-FABP/FABP7), myelin (M-FABP/FABP8), and testis (T-FABP/FABP9) isoforms and each FABP member has a different ligand selectivity and binding affinity for fatty acids due to their structural differences (see, e.g., Furuhashi M, Hotamisligil G S: Fatty acid binding proteins: role in metabolic diseases and potential as drug targets. Nat Rev Drug Discov, 2008; 7:489-503). For example, FABP4, also known as adipocyte FABP (A-FABP), is mainly expressed in adipocytes and macrophages and plays important roles in the development of insulin resistance and atherosclerosis and its expression has been reported to be associated with the pathogenesis of several diseases, predominantly in diabetes mellitus (see, e.g., Furuhashi F. Fatty Acid-Binding Protein 4 in Cardiovascular and Metabolic Diseases. The official journal of the Japan Atherosclerosis Society and the Asian Pacific Society of Atherosclerosis and Vascular Diseases. 2019; 26:216-232). In this regard, FABP4 but not FABP3 was reported to be positively associated with the risk of diabetes which demonstrates the distinct roles and functions of different FABPs within the FABP family (see, e.g., Djousse L and Gaziano JM. Plasma levels of FABP4, but not of FABP3, are associated with increased risk of diabetes. Lipids (2012); 47(8): 757-762). FABP3 is mainly expressed in cardiac myocytes and also in skeletal muscle, kidney mammary glands, testes lungs and stomach (see, e.g., Watanabe K, Wakabayashi H, Veerkamp J H, et al. Immunohistochemical distribution of heart-type fatty acid-binding protein immunoreactivity in normal human tissues and in acute myocardial infarct. J Pathol. 1993;170(1): 59-65.; Zschiesche-Appie Kleine W H, Spitzer Jacques Veerkamp-Jan F C, Glatz E H, et al. Histochemical localization of heart-type fatty-acid binding protein in human and murine tissues. Histochem Cell Biol. 1995;103(2): 147-156.). The normal plasma level of FABP3 in human healthy individuals is reported to be very low compared to the level of FABP3 in human plasma after onset of acute myocardial injury and infarction (see, e.g., Kleine AH, Glatz JFC, Frans A, Van Nieuwenhoven and Van der Vusse GJ. Release of heart fatty acid-binding protein into plasma after acute myocardial infarction in man. Molecular and Cellular Biochemistry 1992; 116:155-162). A cardiac injury can be assessed based on the general health condition of a subject and includes methods known in the art such as electrocardiogram, echocardiogram, angiography or blood tests. Further, a cardiac injury can be assessed by determining the amount of a cardiac injury marker such as NT-proBNP, GDF-15 or troponin. It is envisaged in some embodiments that the subject suffering from a cardiovascular disease as disclosed herein does not suffer from a cardiac injury, particularly an acute cardiac injury, which would result in a higher amount of FABP3 compared to a healthy individual. The skilled person knows that an acute cardiac injury results in a higher FABP3 level in plasma compared to the one in healthy subjects and is in a position to determine an acute cardiac injury in a subject. Accordingly, in a subject suffering from a cardiovascular disease and having an acute cardiac injury, the skilled person is aware of an increased level of FABP3 resulting from a cardiac injury and can distinguish the amount of FABP3 resulting from damaged cells (i.e., cardiac injury) from the effect induced by SGLTi. Particularly, the skilled person knows how to distinguish the amount of FABP3 resulting from damaged cells (i.e., cardiac injury) from the effect induced by SGLTi since the skilled person is aware that the amount of FABP3 resulting from the effect induced by SGLTi is associated with, for example, an increased metabolic function, higher lipid turnover, better microcirculation by comparison with a second marker of myocardial injury that is not involved in the metabolic pathways, but only a structural protein, such as troponin. For example, an increase of the amount of FABP3 in a subject treated with SGLTi but not showing signs of an acute cardiac injury such as elevated troponin indicates that the increase of FABP3 is not resulting from cardiac injury, but as a result from the additional effect induced by SGLTi, such as altered metabolic function, lipid turnover, and/or better microcirculation. Further, the time course of the clinical signs and markers in a subject can be determined by methods known in the art and as disclosed herein such as by an increase or decrease of the amount of a cardiac injury marker such as NT-proBNP, GDF-15 or troponin which indicates worsening by an increase of the amount of a cardiac injury marker such as NT-proBNP, GDF-15 or troponin. To this end, determining worsening of a cardiac injury, e.g., by increase of a cardiac injury marker, can be differentiated by comparison with the change of the amount of FABP3. If an amount of FABP3 is determined in the method of the invention that exceeds said increase of the amount corresponding to the worsening of a cardiac disease, the exceeding amount of FABP3 is indicative for the effect induced by SGLTi.

Accordingly, the amount of FABP3 and a worsening or amelioration of a cardiac injury can be monitored over time to track the relation of the amount of FABP3 corresponding to the worsening or amelioration of the cardiac injury. Thus, in some embodiments of the method of the invention, the amount of FABP3 and optionally of a cardiac injury marker is monitored over time to detect the effect induced by SGLTi. Preferably, the amount of FABP3 and optionally of a cardiac injury marker is monitored for at least 1 hour, at least 1 day, at least 1 week, at least 2 weeks or at least 4 weeks, more preferably for 1 hour, 1 day, 1 week, 2 weeks or 4 weeks. For example, in a subject suffering from a cardiovascular disease according to the present invention, the amount of FABP3 is monitored, wherein an amount of FABP3 higher than the amount of FABP3 corresponding to the worsening of the cardiac injury (as determined by methods known in the art or by a cardiac injury marker) is indicative for the effect induced by SGLTi. In this regard, the skilled person knows that if the amount of FABP3 monitored is equal or not higher than the amount of FABP3 corresponding to the worsening of the cardiac injury, it is indicative that the amount of FABP3 results from a cardiac injury. Accordingly, as disclosed herein above, determining, and preferably monitoring, the amount of FABP3 and a cardiac injury, e.g., by determining a marker for cardiac injury, simultaneously allows for distinguishing the amount of FABP3 resulting from the effect induced by SGLTi from the one resulting from the cardiac injury.

The term “detecting” as used herein, generally, refers to using the information or data generated relating to the amount of FABP3 as referred to herein in a sample of a subject to detect the effect induced by SGLTi The term “effect induced by SGLTi” as used herein refers to an effect that is resulting from the action by SGLTi in a subject suffering from a cardiovascular disease. Particularly, as disclosed herein above, said effect correlates with the amount of FABP3 in a subject, e.g., the effect can be induced by SGLTi via a fasting-like metabolic paradigm involving the metabolic switch from carbohydrates to lipid utilisation and ketogenesis. As also disclosed herein, the term “detecting” may also be seen as “visualizing” or “assessing” the effect(s) induced a SGLTi in a subject suffering from a cardiovascular disease. As mentioned before-FABP3 may be for example be used to assess/visualize whether SGLTi is already active in a subject suffering from a cardiovascular disease; or is still active in a subject suffering from a cardiovascular disease; or is at all active in said subject suffering from a cardiovascular disease, etc.

It is envisaged in accordance with the aforementioned method of the present invention that the subject shall be a subject having a cardiovascular disease. The term “subject” or “patient” as interchangeably used herein may relate to an animal, preferably a mammal, and, more preferably, a human. Particularly, a “patient” or “subject” as used herein may be any single human subject eligible for treatment who is experiencing or has experienced one or more signs, symptoms, or other indicators of a cardiovascular disease, in particular heart failure. Said subject shall exhibit symptoms accompanied herewith, i.e., being at least suspect to suffer from a cardiovascular disease. A “cardiovascular disease” as used herein and in accordance with the present invention relates to any clinical manifestation of a disease state associated with the heart, heart valves and vasculature (e.g., veins and arteries) of the body and encompasses diseases and conditions including but not limited to heart failure, particularly chronic heart failure, arteriosclerosis, atherosclerosis, coronary heart disease, cardiomyopathy, myocardial infarction, acute coronary syndrome, angina, aortic aneurysm, aortic dissection, iliac or femoral aneurysm, pulmonary embolism, primary hypertension, atrial fibrillation, stroke, transient ischemic attack, systolic dysfunction, diastolic dysfunction, myocarditis, atrial tachycardia, ventricular fibrillation, endocarditis, peripheral vascular disease, coronary artery disease (CAD), peripheral artery disease (PAD), and cerebrovascular disease. In some examples, the subject having a cardiovascular disease may also have diabetes, particularly type 2 diabetes. In some embodiments, the subject having a cardiovascular disease in the context of the invention is a subject having heart failure. The term “heart failure” as used herein is a condition that can result from any structural or functional cardiac disorder that impairs the ability of the heart to fill with or pump a sufficient amount of blood throughout the body. Signs and symptoms of heart failure are well known in the art. Particularly, heart failure relates to a diastolic dysfunction or, in particular, to a systolic dysfunction of the heart being accompanied by overt signs of heart failure as known to the person skilled in the art. Preferably, heart failure referred to herein is chronic heart failure (which preferably is caused by systolic dysfunction). The term “chronic heart failure” as used herein refers to chronic, i.e., permanent, heart failure. Heart failure is characterized by an impaired diastolic or systolic blood flow rate and, thus, by an impaired function of the heart. However, rather than exhibiting sudden ischemia accompanied by severe necrosis of the heart muscle cells, chronic heart failure as referred to herein is, preferably, accompanied by continuous necrotic events in heart muscle cells which result in a continuously developing impaired function of the heart. In some embodiments, the subject has systolic or diastolic heart failure. Heart failure according to the present invention includes overt and/or advanced heart failure. In overt heart failure, the patient shows symptoms of heart failure as known to the person skilled in the art. Heart failure can be classified into various degrees of severity. According to the NYHA (New York Heart Association) classification, heart failure patients are classified as belonging to NYHA classes I, II, III and IV. Patients of NYHA Class I have no obvious symptoms of cardiovascular disease but already have objective evidence of functional impairment. Patients of NYHA class Il have slight limitation of physical activity. Patients of NYHA class III show a marked limitation of physical activity. Patients of NYHA class IV are unable to carry out any physical activity without discomfort. They show symptoms of cardiac insufficiency at rest. This functional classification is supplemented by the more recent classification by the American College of Cardiology and the American Heart Association (see J. Am. Coll. Cardiol. 2001;38;2101-2113, updated in 2005, see J. Am. Coll. Cardiol. 2005;46; el-e82). 4 stages A, B, C and D are defined. Stages A and B patients are best defined as those with risk factors for the development of heart failure. For example, patients with coronary artery disease, hypertension, or diabetes mellitus who do not yet demonstrate impaired left ventricular (LV) function, hypertrophy, or geometric chamber distortion would be considered stage A, whereas patients who are asymptomatic but demonstrate LV hypertrophy and/or impaired LV function would be designated as stage B. Stage C then denotes patients with current or past symptoms of heart failure associated with underlying structural heart disease, and stage D designates patients with truly refractory heart failure. In some embodiments, the subject having a cardiovascular disease has heart failure classified as stage B, C or D according to the ACC/AHA classification, and/or wherein the subject has heart failure according to class Il to IV of the NYHA classification. In some embodiments, the subject having a cardiovascular disease has a heart failure with impaired left ventricular ejection fraction (LVEF). Left ventricular ejection fraction (LVEF) can be classified into the categories of heart failure with preserved (HFpEF; LVEF≥50%), mid-range (HFmrEF; LVEF 40-49%), and reduced ejection fraction (HFrEF; LVEF<40%). HFpEF is predominantly characterized by diastolic dysfunction and often results from heart damage due to comorbidities (e.g., obesity, chronic kidney disease (CKD), chronic obstructive pulmonary disease) or accumulation disorders (e.g., cardiac amyloidosis). HFrEF, on the other hand, is characterized prevalently by systolic dysfunction, secondary to a direct heart damage (such as an acute coronary syndrome), a cardiomyopathy or a valve disease. Thus, in some examples, the subject may have a heart failure with reduced ejection fraction (HFrEF), with mildly reduced ejection fraction (HRMEF) or preserved ejection fraction (HFpEF).

The term “sample” as used herein refers to a sample of a bodily fluid, to a sample of separated cells or to a sample from a tissue or an organ. Samples of body fluids can be obtained by well-known techniques. Tissue or organ samples may be obtained from any tissue or organ by, e.g., biopsy. Separated cells may be obtained from the body fluids or the tissues or organs by separating techniques such as centrifugation or cell sorting. Preferably, cell-, tissue-, or organ samples are obtained from those cells, tissues or organs which express or produce the polypeptides referred to herein. The sample may be frozen, fresh, fixed (e.g., formalin fixed), centrifuged, and/or embedded (e.g., paraffin embedded), etc. The cell sample can, of course, be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the amount of the marker in the sample. Likewise, biopsies may also be subjected to post-collection preparative and storage techniques, e.g., fixation. In some embodiments, the sample is a blood, serum, plasma or urine sample, more preferably, the sample is a of blood, serum or plasma sample. It is, in particular, contemplated to measure the level of the biomarker in a plasma sample.

In the context of the present invention, the level of the amount of FABP3 is determined in a sample from a subject having a cardiovascular disease. In some embodiments, additionally, the amount of a cardiac injury marker is determined. Thus, the amount of a single marker can be measured, or the combination of markers comprising the marker FABP3. In all embodiments of the present invention, the amount/level of the respective markers (e, g., biomarkers) or polypeptides used therein (FABP3, cardiac injury marker) are determined by methods known to the person skilled in the art. Preferably, the amount of human marker(s) or polypeptide(s) is/are determined. Determining the amount of the respective marker(s) or polypeptide(s) as referred to herein relates to measuring the amount or concentration, preferably semi-quantitatively or quantitatively. The terms “measuring”, “detecting” and “determining” are used interchangeably herein and relate to the quantification of the marker or polypeptide, e.g., to determining the level of the marker or polypeptide in the sample, employing appropriate methods of detection described herein. Measuring can be done directly or indirectly. Direct measuring relates to measuring the amount or concentration of the marker or polypeptide based on a signal which is obtained from the marker or polypeptide itself and the intensity of which directly correlates with the number of molecules of the marker or polypeptide present in the sample. Such a signal-sometimes referred to herein as intensity signal may be obtained, e.g., by measuring an intensity value of a specific physical or chemical property of the marker or polypeptide. Indirect measuring includes measuring of a signal obtained from a secondary component (i.e., a component not being the marker or polypeptide itself) or a biological read out system, e.g., measurable cellular responses, ligands, labels, or enzymatic reaction products.

In accordance with the present invention, determining the amount of a marker or polypeptide can be achieved by any suitable means and methods for determining the amount of a marker or polypeptide in a sample. Said means and methods comprise immunoassay devices and methods which may utilize labeled molecules in various sandwich, competition, or other assay formats. It is generally known to the skilled person which of the following methods are suitable for qualitative and/or for quantitative detection of a biomarker. Samples can be conveniently assayed for, e.g., proteins using Westerns and immunoassays, like ELISAs, RI-As, fluorescence-based immunoassays, which are commercially available. Further suitable methods comprise measuring a physical or chemical property specific for the marker or polypeptide such as its precise molecular mass or NMR spectrum. Said methods comprise, preferably, biosensors, optical devices coupled to immunoassays, biochips, analytical devices such as mass-spectrometers, NMR-analyzers, or chromatography devices. Further, methods include micro-plate ELISA-based methods, fully-automated or robotic immunoassays (available for example on Elecsys analyzers), CBA (an enzymatic Cobalt Binding Assay, available for example on Roche-Hitachi™ analyzers), and latex agglutination assays (available for example on Roche-Hitachi™ analyzers). Preferably, determining the amount of a marker or polypeptide comprises the steps of (a) contacting a cell capable of eliciting a cellular response the intensity of which is indicative of the amount of the marker or polypeptide with the said marker or polypeptide for an adequate period of time, and (b) measuring the cellular response. For measuring cellular responses, the sample or processed sample is, preferably, added to a cell culture and an internal or external cellular response is measured. The cellular response may include the measurable expression of a reporter gene or the secretion of a substance, e.g., a peptide, polypeptide, or a small molecule. The expression or substance shall generate an intensity signal which correlates to the amount of the marker or polypeptide. Also preferably, determining the amount of a marker or polypeptide comprises the step of measuring a specific intensity signal obtainable from the marker or polypeptide in the sample. As described above, such a signal may be the signal intensity observed at an m/z variable specific for the peptide or polypeptide observed in mass spectra or an NMR spectrum specific for the peptide or polypeptide. Determining the amount of a marker or polypeptide may, preferably, comprises the steps of (a) contacting the marker or polypeptide with a specific ligand, (b) (optionally) removing non-bound ligand, (c) measuring the amount of bound ligand. The bound ligand will generate an intensity signal. Binding according to the present invention includes both covalent and non-covalent binding. A ligand according to the present invention can be any compound, e.g., a peptide, polypeptide, nucleic acid, or small molecule, binding to the peptide or polypeptide described herein. Methods to prepare such ligands are well-known in the art.

The amount of a marker or polypeptide may be, also preferably, determined as follows: (a) contacting a solid support comprising a ligand for the marker or polypeptide as specified above with a sample comprising the marker or polypeptide and (b) measuring the amount of the marker or polypeptide which is bound to the support. The ligand, preferably chosen from the group consisting of nucleic acids, peptides, polypeptides, antibodies and aptamers, is preferably present on a solid support in immobilized form. Materials for manufacturing solid supports are well known in the art and include, inter alia, commercially available column materials, polystyrene beads, latex beads, magnetic beads, colloid metal particles, glass and/or silicon chips and surfaces, nitrocellulose strips, membranes, sheets, duracytes, wells and walls of reaction trays, plastic tubes etc. The ligand or agent may be bound to many different carriers. Examples of well-known carriers include glass, polystyrene, polyvinyl chloride, polypropylene, polyethylene, polycarbonate, dextran, nylon, amyloses, natural and modified celluloses, polyacrylamides, agaroses, and magnetite. The nature of the carrier can be either soluble or insoluble for the purposes of the invention. Suitable methods for fixing/immobilizing said ligand are well known and include, but are not limited to ionic, hydrophobic, covalent interactions and the like.

The term “amount” as used herein encompasses the absolute amount of a marker as referred to herein, such as FABP3 or NT-proBNP, the relative amount or concentration of the said biomarker as well as any value or parameter which correlates thereto or can be derived therefrom. Such values or parameters comprise intensity signal values from all specific physical or chemical properties obtained from the said marker(s) by direct measurements, e.g., intensity values in mass spectra or NMR spectra. Moreover, encompassed are all values or parameters which are obtained by indirect measurements as described herein, e.g., response amounts determined from biological read out systems in response to the peptides or intensity signals obtained from specifically bound ligands. It is to be understood that values correlating to the aforementioned amounts or parameters can also be obtained by all standard mathematical operations.

The term “comparing” as used herein refers to comparing the amount of the marker, such as FABP3 or NT-proBNP, in the sample from the subject or patient with the reference amount of the biomarker as described herein. It is to be understood that comparing as used herein usually refers to a comparison of corresponding parameters or values, e.g., an absolute amount is compared to an absolute reference amount while a concentration is compared to a reference concentration or an intensity signal obtained from the marker in a sample is compared to the same type of intensity signal obtained from a reference sample. The comparison may be carried out manually or computer assisted. Thus, the comparison may be carried out by a computing device. The value of the measured or detected amount of the biomarker in the sample from the individual or patient and the reference level can be, e.g., compared to each other and the said comparison can be automatically carried out by a computer program executing an algorithm for the comparison. The computer program carrying out the said evaluation will provide the desired assessment in a suitable output format. For a computer assisted comparison, the value of the determined amount may be compared to values corresponding to suitable references which are stored in a database by a computer program. The computer program may further evaluate the result of the comparison, i.e., automatically provide the desired assessment in a suitable output format. For a computer assisted comparison, the value of the determined amount may be compared to values corresponding to suitable references which are stored in a database by a computer program. The computer program may further evaluate the result of the comparison, i.e., automatically provides the desired assessment in a suitable output format. Based on the comparison of the amount determined in step a) and a reference amount, it is possible to detect the effect induced by SGLTi. Therefore, the reference amount is to be chosen so that either a difference or a similarity in the compared amounts allows detecting the effect induced by SGLTi according to the present invention.

The “reference amount” as used herein and to be applied in the context of the present invention preferably refers to a determined or predetermined value obtained from a sample of a subject or from samples from a group of subjects who suffer from a cardiovascular disease. Preferably, the reference amount is determined or has been predetermined in a reference sample from the disease entity to which the patient belongs. Therefore, the reference amount will in general be derived from a subject known to have a cardiovascular disease. For example, in case that the subject suffers from heart failure, the reference amount is determined or has been predetermined in a sample from a subject that shall also suffer from heart failure. It is contemplated that the extend of a cardiovascular disease, such as heart failure, of the subject is essentially the same as of the reference subject. The extent or severity of a cardiovascular disease, such as heart failure, can be determined by a person skilled in the art without further ado and is also disclosed herein above. Such a reference amount can be, e.g., a threshold amount wherein an amount larger than the threshold shall be indicative for the effect induced by SGLTi. In some examples, the reference amount refers to an amount of FABP3 before the subject is treated with SGLTi. Particularly, in some examples, the reference amount of FABP3 before the subject is treated with SGL of FABP3 is about 37.8 ng/ml. Preferably, the term “about” as used herein encompasses a range of +and −20%, more preferably a range of +and −10%, even more preferably a range of +and −5%, and most preferably a range of +and −2%, relative to the specific amount, e.g., indication of an amount of “about 100” is meant to encompass an amount within a range from 80 to 120. Also, the term “about” refers to the exact amount. Preferably, the amount or levels are measured as described in the Examples. In some embodiments, the reference amount is the upper limit of normal (ULN) of the amount in samples of subjects suffering from a cardiovascular disease. The ULN for samples of subjects, i.e., a given population of subjects, can be determined by various well-known techniques. In this context, the skilled person knows how to set the reference amount to meet routine requirements. The reference amount can e.g., be set to any percentage between 25% and 75% or be set to the median, tertiles or quartiles as determined from the overall distribution of the values in reference samples to be determined. Typically, the reference sample is from essentially the same type of cells, tissue, organ or body fluid source as the sample from the subject or patient subjected to the method of the invention, e.g., if according to the invention blood is used as a sample to determine the amount of the biomarker in the individual, the reference amount is also determined in blood or a part thereof.

In some examples, the reference amount may comprise a reference amount of FABP3 and a reference amount of a cardiac injury marker. Particularly, in some embodiments, the method comprises forming a ratio between the amount of FABP3 and the amount of a cardiac injury marker and comparing the ratio to a reference ratio. For example, the cardiac injury marker is a cardiac function marker, a cardiac necrosis marker or a cardiac inflammatory marker. A threshold reference amount or a reference ratio can be calculated without further ado by statistical tests in a sample from a subject suffering from a cardiovascular disease and/or from a group of subjects suffering from a cardiovascular disease, e.g., a reference amount can be determined from Receiver-Operator-Curves (ROC) according to clinical studies and as described herein. For example, the reference amount may be determined in a reference sample from a subject or a group of subjects having a cardiovascular disease, such as a heart failure, and which are treated or not treated with SGLTi. For example, the reference amount can be determined in a subject being treated with SGLTi for use in monitoring the effect induced by SGLTi. As another example, the reference amount can be determined in a subject not being treated with SGLTi, e.g., for quantifying the effect induced by SGLTi in said subject. In some embodiments, the reference amount is the amount in a sample of a subject suffering from a cardiovascular disease or the ULN of the amount in samples of subjects suffering from a cardiovascular disease before being treated with SGLTi. Particularly, it is envisaged that the subject and the reference subject or group thereof have essentially the same reference amount of FABP3. Whether two amounts are essentially the same can be assessed by the skilled person without further ado. For example, amounts which differ by less than 5% or less than 3% are regarded to be essentially the same.

In some embodiments, the sodium-glucose cotransporter inhibitor (SGLTi) is a sodium-glucose cotransporter 2 inhibitor (SGLT2i) or a sodium-glucose cotransporter 1 inhibitor (SGLT1i), preferably a SGLT2i, more preferably a SGLT2i, more preferably a gliflozin, more preferably the SGLTi comprises canagliflozin, dapagliflozin, and empagliflozin, ertugliflozin, ipragliflozin, remogliflozin, sergliflozin, sotagliflozin or tofogliflozin. Current available SGLT inhibitors are developed based on the natural product phlorizin and include, for example, but are not limited to SGLT2 inhibitors, such as empagliflozin, dapagliflozin, and canagliflozin; SGLT1 inhibitors, such as KGA-2727 and mizagliflozin; and dual inhibitors which inhibit both SGLT1 and SGLT2, such as sotagliflozin and LX2761. Particularly, sodium-glucose cotransporter 2 (SGLT2) are high-capacity, low-affinity cotransporter located in the renal proximal tubular epithelium and mainly reabsorb filtered glucose (Bakris G L, Fonseca V A, Sharma K, Wright E M. Renal sodium-glucose transport: role in diabetes mellitus and potential clinical implications. Kidney international. 2009;75(12) 1272-7). Besides the renal specific effects on glycemic control, SGLT2 are responsible for=5% of sodium reabsorption at the proximal tubule under normal conditions. In the setting of chronic hyperglycemia, the capacity of SGLT2 in the kidney is increased leading to more pronounced effects on sodium homeostasis (Bakris G L, Fonseca V A, Sharma K, Wright E M. Renal sodium-glucose transport: role in diabetes mellitus and potential clinical implications. Kidney international. 2009; 75(12):1272-7). Following, inhibition of SGLT2 increases diuresis and decreases plasma volume and blood pressure (Baker W L, Smyth L R, Riche D M, Bourret E M, Chamberlin K W, White W B. Effects of sodium-glucose co-transporter 2 inhibitors on blood pressure: a systematic review and metaanalysis. Journal of the American Society of Hypertension: JASH. 2014;8(4):262-75.e9). SGLT2i have been shown to exert cardioprotective and nephroprotective effects by promoting autophagic flux and mitigating apoptosis, muting oxidative and other cellular stresses, inhibiting proinflammatory and profibrotic pathways, and enhancing cellular energy stores and metabolism. Therefore, it has been reported that SGLT2i improve cardiovascular outcomes in patients, including those with heart failure, such as with a reduced or preserved ejection fraction. It is envisaged by the method of the present invention that SGLTi is administered to a subject suffering from a cardiovascular disease. The term “administering” as used herein is used in the broadest sense and inter alia encompasses oral, enteral, topical administration and “parenteral administration”. “Parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intrastemal injection and infusion.

The term “FABP3” as used herein refers to the heart fatty acid binding protein, in particular to the human heart fatty acid binding protein. Preferably, the term also includes variants of the heart type fatty acid binding protein. FABP3 is frequently also referred to heart type fatty acid binding protein. FABP3 is also known as H-FABP. The cDNA sequence as well the protein sequence of human FABP3 is well known in the art and was first described by Peters et al. (Biochem. J. 276 (Pt 1), 203-207 (1991)). Moreover, the sequence of human H-FABP can be found, preferably, in Genebank entry U57623.1 (cDNA sequence) and AAB02555.1 (protein sequence). The major physiological of function of FABP3 is thought to be the transport of free fatty acids, see e.g., Storch et al., Biochem. Biophys. Acta. 1486(2000), 28-44. FABP3 as used herein encompasses also variants of the aforementioned specific FABP3 polypeptides. Such variants have at least the same essential biological properties as the specific FABP3 polypeptide. In particular, they share the same essential biological properties if they are detectable by the same specific assays referred to in this specification, e.g., by ELISA assays using polyclonal or monoclonal antibodies specifically recognizing the said FABP3 polypeptides. A preferred assay is described in the accompanying Examples. Moreover, it is to be understood that a variant as referred to in accordance with the present invention shall have an amino acid sequence which differs due to at least one amino acid substitution, deletion and/or addition wherein the amino acid sequence of the variant is still, preferably, at least about 50%, at least about 60%, at least about 70%, at least about 80%, at least about 85%, at least about 90%, at least about 92%, at least about 95%, at least about 97%, at least about 98%, or at least about 99% identical with the amino sequence of the specific FABP3 polypeptides, preferably with the amino acid sequence of human FABP3, more preferably over the entire length of the specific FABP3, e.g. of human FABP3. The degree of identity between two amino acid sequences can be determined as described above. Variants referred to above may be allelic variants or any other species specific homologs, paralogs, or orthologs. Moreover, the variants referred to herein include fragments of the specific FABP3 polypeptides or the aforementioned types of variants as long as these fragments have the essential biological properties as referred to above. Such fragments may be, e.g., degradation products of the FABP3 polypeptides. Further included are variants which differ due to posttranslational modifications such as phosphorylation or myristylation.

In some embodiments, the present invention further comprises additionally determining the amount of a cardiac injury marker. A cardiac injury marker as used herein is indicative of myocardial damage, injury or necrosis, e.g., wherein the muscle mass is reduced, meaning that the heart has a reduced contractile capacity. In some examples, the cardiac injury marker preferably is a cardiac function, a cardiac inflammatory or a cardiac necrosis marker. The preferred cardiac injury marker is a cardiac function marker such as NT-proBNP or a cardiac necrosis marker such as cardiac troponin. In some examples, an increased amount of FABP3 and the absence of an increase of the amount of the cardiac injury marker compared to a reference amount is indicative for the effect induced by SGLTi. Whether the cardiac injury marker is increased or not increased can be assessed by the skilled person without further ado. For example, amounts which differ by less than 5% or less than 3% are regarded to be not increased. A cardiac function marker as used herein is indicative of a malfunction of the myocard, i.e., the muscle tissue in the myocardium is weaker than normal and cannot contract as does healthy tissue, meaning that the heart has to perform harder than normal to ensure a sufficient blood supply to the body. A cardiac necrosis marker as used herein indicates cell death having occurring in the myocard of the subject, which can occur after prolonged states of ischemia or as a consequence of apoptosis. An inflammatory marker as used herein is indicative of inflammatory processes occurring in the body, particularly in the myocard, of the individual. In one embodiment, the cardiac marker is a cardiac function marker, preferably wherein the cardiac function marker is a natriuretic peptide, more preferably BNP or NT-proBNP, more preferably NT-proBNP. The term “natriuretic peptide” comprises Atrial Natriuretic Peptide (ANP)-type and Brain Natriuretic Peptide (BNP)-type peptides and variants thereof. BNPtype peptides comprise pre-proBNP, proBNP, NT-proBNP, and BNP (see, e.g., Bonow, 1996, Circulation 93: 1946-1950; WO 02/089657, WO 02/083913). The aforementioned pre-pro peptide of the brain natriuretic peptide (having 134 amino acids in length) comprises a short signal peptide, which is enzymatically cleaved off to release to pro peptide (108 amino acids). The pro peptide is further cleaved into an N-terminal pro peptide (NT-pro peptide, 76 amino acids) and the active hormone (32 amino acids). Preferably, a natriuretic peptide according to the present invention is BNP or NT-proBNP or a variant thereof. The most preferred BNP-type peptide referred to herein is human NT-proBNP. The conventional diagnostic techniques for heart failure are based on the well known vascular volume stress marker NT-proBNP. As briefly discussed above, the human NT-proBNP, as referred to in accordance with the present invention, is a polypeptide comprising, preferably, 76 amino acids in length corresponding to the N-terminal portion of the human NT-proBNP molecule. The structure of the human BNP and NT-proBNP has been described already in detail in the prior art, e.g., WO 02/089657, WO 02/083913. Preferably, human NT-proBNP as used herein is human NT-proBNP as disclosed in EP 0 648 228 B1. The NT-proBNP referred to in accordance with the present invention further encompasses allelic and other variants of said specific sequence for human NT-proBNP discussed above. Specifically, envisaged are variant polypeptides which are on the amino acid level at least 60 % identical, more preferably at least 70 %, at least 80 %, at least 90 %, at least 95 %, at least 98% or at least 99 % identical, to human NT-proBNP. Substantially similar and also envisaged are proteolytic degradation products which are still recognized by the identification means or by ligands directed against the respective full-length peptide. Also encompassed are variant polypeptides having amino acid deletions, substitutions, and/or additions compared to the amino acid sequence of human NT-proBNP as long as the said polypeptides have NT-proBNP properties. NT-proBNP properties as referred to herein are immunological and/or biological properties. Preferably, the NT-proBNP variants have immunological properties (i.e. epitope composition) comparable to those of NT-proBNP. Thus, the variants shall be recognizable by the aforementioned means or ligands used for determination of the amount of the natriuretic peptides. Biological and/or immunological NT-proBNP properties can be detected by the assay described in Karl et al. (Karl 1999, Scand J Clin Invest 59:177-181), Yeo et al. (Yeo 2003, Clinica Chimica Acta 338:107-115). Variants also include posttranslationally modified peptides such as glycosylated peptides. Further, a variant in accordance with the present invention is also a peptide or polypeptide which has been modified after collection of the sample, for example by covalent or non-covalent attachment of a label, particularly a radioactive or fluorescent label, to the peptide. For example, a cardiac necrosis marker can be a cardiac troponin. The term “cardiac troponin” as used herein refers to all troponin isoforms expressed in cells of the heart. These isoforms are well characterized in the art as described, e.g., in Anderson 1995, Circulation Research, vol. 76, no. 4:681-686 and Ferrieres 1998, Clinical Chemistry, 44:487-493. Patients suffering from myocardial infarction can be diagnosed using cardiac troponins, preferably troponin T or I. Myocardial infarction is regarded as being caused by a necrotic state of the myocard, i.e., cell death. Cardiac troponins are released following cell death and can hence be used for the diagnosis of myocardial infarction. If the amount of Troponin T in the blood is elevated, i.e. above 0.1 ng/ml, an acute cardiovascular event is assumed and the patent is treated accordingly. Preferably, cardiac Troponin refers to Troponin T or Troponin I. It is to be understood that isoforms of Troponins may be determined in the method of the present invention together, i.e. simultaneously or sequentially, or individually, i.e. without determining the other isoform at all. Amino acid sequences for human Troponin T and human Troponin I are disclosed in Anderson, loc cit and Ferrieres 1998, Clinical Chemistry, 44:487-493. The term “cardiac Troponin” encompasses also variants of the aforementioned specific Troponins. Such variants have at least the same essential biological and immunological properties as the specific cardiac Troponins. In particular, they share the same essential biological and immunological properties if they are detectable by the same specific assays referred to in this specification, e.g., by ELISA Assays using polyclonal or monoclonal antibodies specifically recognizing the said cardiac Troponins. Moreover, it is to be understood that a variant as referred to in accordance with the present invention shall have an amino acid sequence which differs due to at least one amino acid substitution, deletion and/or addition wherein the amino acid sequence of the variant is still, preferably, at least about 50%, at least about 60%, at least about 70%, at least about 80%, at least about 85%, at least about 90%, at least about 92%, at least about 95%, at least about 97%, at least about 98%, or at least about 99% identical with the amino sequence of the specific Troponin. Variants may be allelic variants or any other species specific homologs, paralogs, or orthologs. Moreover, the variants referred to herein include fragments of the specific cardiac Troponins or the aforementioned types of variants as long as these fragments have the essential immunological and biological properties as referred to above. Preferably, the cardiac troponin variants have immunological properties (i.e. epitope composition) comparable to those of human troponin T or troponin I. Thus, the variants shall be recognizable by the aforementioned means or ligands used for determination of the amount of the cardiac troponins. Thus, the variants shall be recognizable by the aforementioned means or ligands used for determination of the amount of the cardiac troponins. Such fragments may be, e.g., degradation products of the Troponins. Further included are variants which differ due to posttranslational modifications such as phosphorylation or myristylation. Preferably the biological property of troponin I and its variant is the ability to inhibit actomyosin ATPase or to inhibit angiogenesis in vivo and in vitro, which may 5 e.g. be detected based on the assay described by Moses et al. 1999 PNAS USA 96 (6): 2645-2650). Preferably the biological property of troponin T and its variant is the ability to form a complex with troponin C and I, to bind calcium ions or to bind to tropomyosin, preferably if present as a complex of troponin C, I and T or a complex formed by troponin C, troponin I and a variant of troponin T. Preferably, the amount of a cardiac troponin, particularly troponin T, is determined with a very sensitive troponin T test system in order to allow a reliable determination of very low cardiac troponin amounts, preferably said test system is capable of determining amounts of 0.002 ng/ml troponin in a sample, preferably, in a blood, blood serum or blood plasma sample. A particularly preferred Troponin T assay is the Elecsys® 2010 analyzer (Roche Diagnostics) with a detection limit of from about 0.001 ng/ml to about 0.0015 ng/ml, in general about 0.0015 ng/ml. The term “Growth-Differentiation Factor-15” or “GDF-15” relates to a polypeptide being a member of the transforming growth factor (TGF) cytokine superfamily. GDF-15 was originally cloned as macrophage-inhibitory cytokine 1 and later also identified as placental transforming growth factor-15, placental bone morphogenetic protein, non-steroidal anti-inflammatory drug-activated gene 1, and prostate-derived factor (Bootcov loc cit; Hromas, 1997 Biochim Biophys Acta 1354:40-44; Lawton 1997, Gene 203:17-26; Yokoyama-Kobayashi 1997, J Biochem (Tokyo), 122:622-626; Paralkar 1998, J Biol Chem 273:13760-13767). Similar to other TGF-related cytokines, GDF-15 is synthesized as an inactive precursor protein, which undergoes disulfide-linked homodimerization. Upon proteolytic cleavage of the N terminal propeptide, GDF-15 is secreted as a ~28 kDa dimeric protein (Bauskin 2000, Embo J 19:2212-2220). Amino acid sequences for GDF-15 are disclosed in WO99/06445, WO00/70051, WO2005/113585, Bottner 1999, Gene 237:105-111, Bootcov loc. cit, Tan loc. cit., Baek 2001, Mol Pharmacol 59:901-908, Hromas loc cit, Paralkar loc cit, Morrish 1996, Placenta 17:431-441 or YokoyamaKobayashi loc cit.

The invention also relates to a computer-implemented method for carrying out the method of any of the preceding claims, comprising a) means for determining the amount of FABP3, or the amount of FABP3 and a the cardiac injury marker, in the sample of a subject treated with SGLTi, wherein the subject suffers from a cardiovascular disease; and b) means for comparing said amount of FABP3 to a reference amount, said amount of FABP3 and said amount of a cardiac injury marker to a reference amount, or means for forming and comparing the ratio between said amount of FABP3 and said amount of the cardiac injury marker to a reference ratio It follows from the above that at least some steps of the method of the invention may be performed by a computing device. The computing device may also have access to an output device. Exemplary output devices include fax machines, displays, printers, and files, for example. According to some embodiments of the present disclosure, a computing device may perform one or more steps of a method disclosed herein, and thereafter provide an output, via an output device, relating to a result, indication, ratio or other factor of the method. It should also be understood that multiple computing devices may be used together, such as over a network or other methods of transferring data, for performing one or more steps of the methods disclosed herein. Exemplary computing devices include desktop computers, laptop computers, cellular devices, tablet computers, servers, and the like. In general, a computing device comprises a processor capable of executing a plurality of instructions such as a program of software.

The invention also relates to a computer program product comprising a program code for executing the computer-implemented method according to the present invention when run on at least one computer. Software as used herein may include instructions which, when executed by a processor of the computing device, may perform one or more steps of the methods disclosed herein. Some of the instructions may be adapted to produce signals that control operation of other machines and thus may operate through those control signals to transform materials far removed from the computer itself. These descriptions and representations are the means used by those skilled in the art of data processing, for example, to most effectively convey the substance of their work to others skilled in the art.

The present invention also relates to a device for carrying out the method of any of the preceding claims, comprising a) means for determining the amount of FABP3, or the amount of FABP3 and a cardiac injury marker, in a sample of a subject treated SGLTi, wherein the subject suffers from a cardiovascular disease; and b) means for comparing said amount of FABP3 to a reference amount, said amount of FABP3 and said amount of a cardiac injury marker to a reference amount, or means for forming and comparing the ratio between said amount of FABP3 and said amount of the cardiac marker to a reference ratio. The term “device” as used herein relates to a system of means comprising at least the aforementioned means operatively linked to each other as to allow detecting the effect induced by SGLTi. Preferred means for determining the amount of FABP3, and optionally the amount of a cardiac injury marker such as NT-proBNP, as well as means for carrying out the comparison are disclosed above in connection with the method of the invention. How to link the means in an operating manner will depend on the type of means included into the device. For example, where means for automatically determining the amount of the biomarkers or polypeptides are applied, the data obtained by said automatically operating means can be processed by, e.g., a computer program in order to obtain the desired results. Preferably, the means are comprised by a single device in such a case. Said device may accordingly include an analyzing unit for the measurement of the amount of the peptides or polypeptides in an applied sample and a computer unit for processing the resulting data for the evaluation. The computer unit, preferably, comprises a database including the stored reference amounts or values thereof recited elsewhere in this specification as well as a computer-implemented algorithm for carrying out a comparison of the determined amounts for the polypeptides with the stored reference amounts of the database. Computer-implemented as used herein refers to a computer-readable program code tangibly included into the computer unit. Alternatively, where means such as test stripes are used for determining the amount of the biomarkers or polypeptides, the means for comparison may comprise control stripes or tables allocating the determined amount to a reference amount. The test stripes are, preferably, coupled to a ligand which specifically binds to the markers or polypeptides referred to herein. The strip or device, preferably, comprises means for detection of the binding of said markers or polypeptides to the said ligand. Preferred means for detection are disclosed in connection with embodiments relating to the method of the invention above. In such a case, the means are operatively linked in that the user of the system brings together the result of the determination of the amount and the diagnostic or prognostic value thereof due to the instructions and interpretations given in a manual. The means may appear as separate devices in such an embodiment and are, preferably, packaged together as a kit. The person skilled in the art will realize how to link the means without further ado. Preferred devices are those which can be applied without the particular knowledge of a specialized clinician, e.g., test stripes or electronic devices which merely require loading with a sample. The results may be given as output of raw data which need interpretation by the clinician. Preferably, the output of the device is, however, processed, i.e., evaluated, raw data the interpretation of which does not require a clinician. Further preferred devices comprise the analyzing units/devices (e.g., biosensors, arrays, solid supports coupled to ligands specifically recognizing the natriuretic peptide, Plasmon surface resonance devices, NMR spectrometers, mass-spectrometers etc.) and/or evaluation units/devices referred to above in accordance with the method of the invention.

The present invention also relates to a kit for carrying out the method of any of the preceding claims, comprising a) means for determining the amount of FABP3, or the amount of FABP3 and a cardiac injury marker, in a sample of a subject treated with SGLTi, wherein the subject suffers from a cardiovascular disease; and b) means for comparing said amount of FABP3 to a reference amount, said amount of FABP3 and said amount of the cardiac injury marker to a reference amount, or means for forming and comparing the ratio between said amount of FABP3 and said amount of the cardiac marker to a reference ratio. The term “kit” as used herein refers to a collection of the aforementioned means, preferably, provided in separately or within a single container. The contained, also preferably, comprises instructions for carrying out the method of the present invention. These instructions may be in the form of a manual or may be provided by a computer program code which is capable of carrying out the comparisons referred to in the methods of the present invention and to establish an identification accordingly when implemented on a computer or a data processing device. Further, the kit shall comprise at least one standard for a reference as defined herein above, i.e., a solution with a reference amount for FABP3 and optionally further markers such as NT-proBNP as referred to herein representing a reference amount.

The present invention also relates to the use of i) FABP3, FABP3 and a cardiac injury marker, and/or ii) use of a detection agent for FABP3, for FABP3 and the cardiac injury marker, for detecting the effect induced by SGLTi in a subject suffering from a cardiovascular disease.

The invention will be further illustrated by the following non-limiting Experimental Examples.

### EXPERIMENTAL EXAMPLES

### Example 1

### Study Design and Ethical Aspects

The study is a prospective single-center clinical trial at the Medical University of Vienna, Department of Internal Medicine II/Division of Cardiology. The study protocol complies with the Declaration of Helsinki and was approved by the ethics committee of the Medical University of Vienna (EK1729/2013).

A written informed consent from all patients was present before inclusion. Patient data collected in this study are anonymized. In the raw and analysis ready datasets, all personally identifiable information are removed or replaced, removing the ability to identify individual participants. Patients non-willing to participate or withdraw informed consent were excluded from the prospective registry.

### Patient Population

A total of 100 patients with stable chronic HFrEF participating in a prospective registry at the heart failure outpatient clinic at the Medical University of Vienna, which has been imputed with all clinically relevant data, are included in the present study. The inclusion criteria of the registry are documented chronic HFrEF, an age of at least 18 years and stable heart failure related medical therapy in the last 3 months. Inclusion criteria for this study is the participation in the registry, and the absence of any exclusion criteria avoiding the prescription of SGLT-2 inhibitor (Dapagliflozin). Visits at the outpatient clinic are conducted at baseline (visit 0), at week 4 after SGLT-2 inhibitor therapy initiation (visit 1), after 3 month (visit 2), after 6 months (visit 3), and every 6 months thereafter.

### Study Endpoints: Primary and Secondary Endpoints

All-cause mortality is defined the primary endpoint. Pre-defined secondary endpoints include hospitalization for any reason and worsening of the disease, assessed by deterioration in “New York Heart Association” (NYHA) functional class, changes in NT-proBNP level and changes in left ventricular ejection fraction (LVEF). Association of the primary and secondary endpoints are tested for the body fluid status at baseline and its changes over time, for blood glucose (including blood glucose and glycated hemoglobin (HbA1c, %)) and for kidney function (creatinine and eGFR).

### Demographics

Information on baseline characteristics, medical history as well as current medications at the time of inclusion and during follow-up, including information on previous coronary angiography, right heart catheterization, echocardiographic data, cardiac death, myocardial infarction, hospitalization due to heart failure, angina pectoris, or implantation of ICD and/or PM and stroke will be recorded.

### Sampling and Laboratory Measurement

Blood and urine samples are drawn from a peripheral vein and collected at baseline prior to administration of the SGLT-2 inhibitor and repeated at every regularly scheduled study-visit. Samples are used for (1.) routine laboratory analysis and (2.) biobank storage.

### Biobank

Blood for the biobank is immediately centrifuged and plasma samples are stored at-80° C. for future analysis. The plasma samples and urine samples are stored in the central “biobank” facility of the Department of Laboratory Medicine at the Medical University of Vienna.

### Routine Laboratory Parameters

Routine laboratory parameters such as blood count, CRP, creatinine or GFR and urinalysis are done according to the local laboratory's standard procedures.

### Biomarker Measurements

FABP3 and additional cardiac biomarkers N-terminal B-type natriuretic peptide (NT-proBNP) and GDF-15 are measured in plasma using the Elecsys Systems (Roche Diagnostics, Mannheim, Germany).

### Bioelectrical Impedance Analysis

Bioelectrical impedance analysis (BIA) for assessment of the body fluid status is performed at inclusion prior to administration of the SGLT-2 inhibitor, as well as at every scheduled study-visit thereafter.

### Echocardiographic Assessment

All patients undergo routine echocardiograms using commercially available equipment (Vivid7, GE-Healthcare). Cardiac morphology is assessed using diameters in four-and two chamber views and ejection fraction is calculated using the biplane Simpson method according to EAE recommendations (Lang RM, Bierig M, Devereux RB, Flachskampf FA, Foster E, Pellikka PA, et al. Recommendations for chamber quantification. European journal of echocardiography: the journal of the Working Group on Echocardiography of the European Society of Cardiology. 2006; 7(2):79-108). Valvular morphology is assessed in a standard view according to the guidelines and quantified by an integrated approach comprising valvular morphology and function (Zoghbi W A, Adams D, Bonow R O, Enriquez-Sarano M, Foster E, Grayburn PA, et al. Recommendations for Noninvasive Evaluation of Native Valvular Regurgitation: A Report from the American Society of Echocardiography Developed in Collaboration with the Society for Cardiovascular Magnetic Resonance. Journal of the American Society of Echocardiography: official publication of the American Society of Echocardiography. 2017; 30(4): 303-71). Systolic pulmonary artery pressures (sPAP) is calculated to the estimated central venous pressure. Raw data with frame rates >50 fps are stored digitally for postprocessing.

### Follow up

Follow-up visits are scheduled as described above. In case of missed outpatient-clinic visit, telephone-visits are done. Blood samples, urine samples, and body fluid measurement are done at each visit. Echocardiographic examination are repeated at visit 3 (6 months after inclusion). Information on functional status, major cardiovascular events and hospitalization due to heart failure are recorded. Furthermore, mortality is determined via retrieval query of the Austrian Death Registry in order to minimize a potential bias in case of losses to follow-up.

### Results

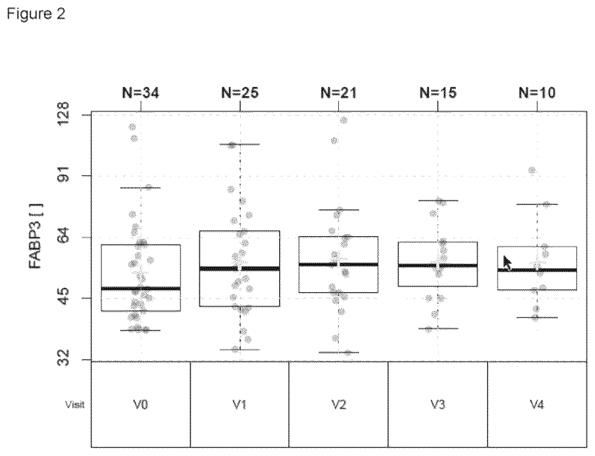

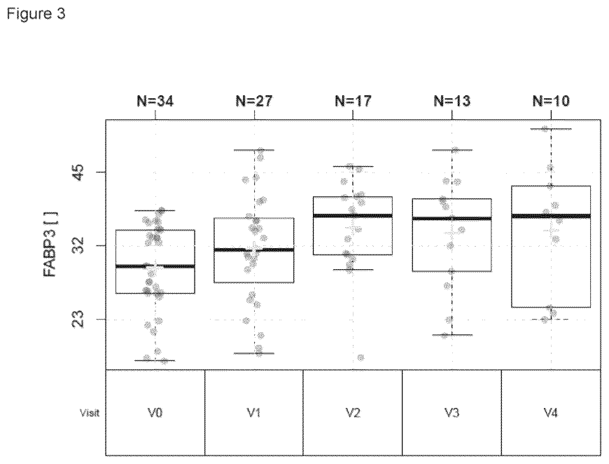

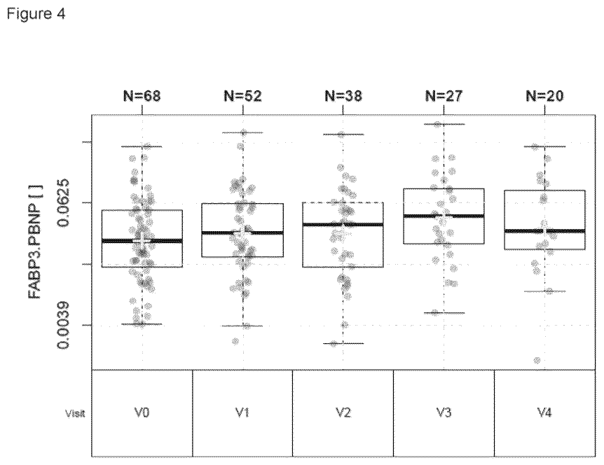

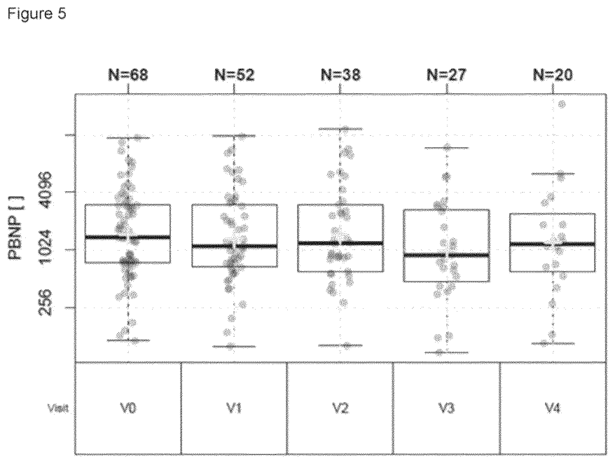

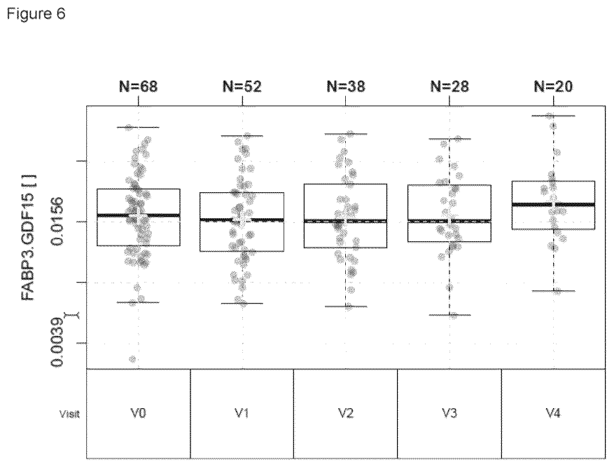

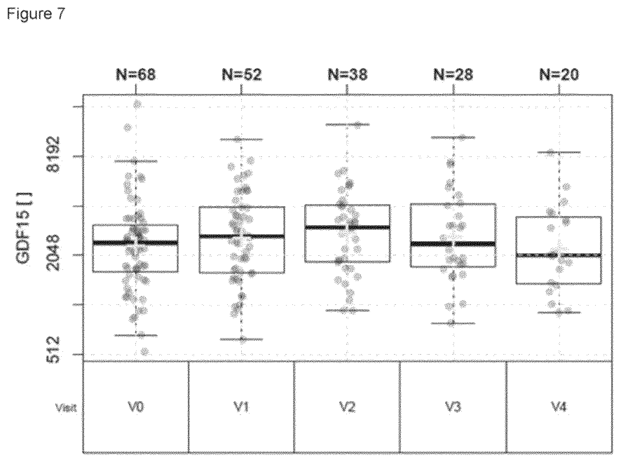

Initiation of SGLT2i therapy is associated with an increase in plasma concentration of FABP3 in patients being susceptible and/or as likely to respond to SGLT2i medication (FIG. 1). As shown in FIG. 2, in the patient subgroup with high baseline FABP3 (above the median concentration of 37.8 ng/ml) the amount of FABP3 at 3 months (V2) is about 14% higher compared to the amount of FABP3 at baseline. As shown in FIG. 3, in the patient subgroup with low baseline FABP3 (below the median concentration of 37.8 ng/ml), the amount of FABP3 at 3 months (V2) is about 27% higher compared to the amount of FABP3 at baseline. The FABP3/NT-proBNP ratio increases over time after SGLT2i initiation, with a peak of relative increase of 77% vs. 19% for the FABP3 concentration after 6 months (V3) alone (FIG. 4). Conversely, the amount of NT-proBNP before and after SGLT2i initiation does not correlate with a patient's susceptibility and/or response to SGLT2i administration (FIG. 5). Further, the FABP3/GDF-15 ratio remains stable after SGLT2i initiation, with a peak of relative increase at 12 months (V4) of 12% (FIG. 6). In this regard, the amount of GDF-15 did not significantly change before and after SGLT2i initiation (FIG. 7).

The invention is also characterized by the following items.
1. A method of detecting the effect induced by a sodium-glucose cotransporter inhibitor (SGLTi) in a subject suffering from a cardiovascular disease, the method comprising a) determining the amount of Fatty Acid Binding Protein 3 (FABP3) in a sample of the subject treated with SGLTi; b) comparing the amount of said FABP3 to a reference amount.
2. The method of item 1, wherein an increased amount of FABP3 compared to the reference amount is indicative for the effect.
3. The method of item 2, wherein an increased amount of FABP3 by at least 10% compared to the reference amount is indicative for the effect.
4. The method of any of item 1-3, wherein the reference amount is the amount in a sample of a subject suffering from a cardiovascular disease.
5. The method of any of items 1-3, wherein the reference amount is the upper limit of normal (ULN) of the amount in samples of subjects suffering from a cardiovascular disease.
6. The method of any of items 1-3, wherein the reference amount is the amount in a sample from the same subject suffering from a cardiovascular disease.
7. The method of any of the preceding items, wherein the reference amount is the amount in a sample of a subject suffering from a cardiovascular disease or the ULN of the amount in samples of subjects suffering from a cardiovascular disease before being treated with SGLTi.
8. The method of any of the preceding items, the method further comprising: c) determining the amount of FABP3 and a cardiac injury marker in a sample of the subject treated with SGLTi and comparing the amount of said FABP3 and the amount of said cardiac injury marker to a reference amount.
9. The method of any of item 8, the method further comprising: d) forming a ratio between the amount of FABP3 and the amount of the cardiac injury marker and comparing the ratio to a reference ratio.
10. The method of item 9, wherein an increased ratio between the amount of FABP3 and the amount of the cardiac injury marker compared to the reference ratio is indicative for the effect.
11. The method of any of the preceding claims, wherein the amount of FABP3, and optionally the amount of a cardiac injury marker, is monitored, preferably wherein the amount of FABP3, and optionally the amount of a cardiac injury marker, is monitored for at least 1 hour, at least 1 day, at least 1 week, at least 2 weeks or at least 4 weeks, more preferably for 1 hour, 1 day, 7 days or 14 days.
12. The method of any of items 8-11, wherein the cardiac injury marker comprises a cardiac function marker.
13. The method of any of the preceding items, wherein the cardiac marker is a natriuretic peptide, more preferably BNP or NT-proBNP, more preferably NT-proBNP.
14. The method of any of items 8-13, wherein the cardiac injury marker is NT-proBNP and a ratio between the amount of FABP3 and the amount of NT-proBNP of at least 0.030, preferably of at least 0.032, more preferably of at least 0.039 is indicative for the effect.
15. The method of any of the preceding items, wherein the subject having a cardiovascular disease has heart failure, optionally chronic heart failure.
16. The method of any of the preceding items, wherein the subject having a cardiovascular disease has systolic or diastolic heart failure.
17. The method of any of the preceding items, wherein the subject having a cardiovascular disease has an impaired left ventricular ejection fraction (LVEF).
18. The method of any of the preceding items, wherein the subject having a cardiovascular disease has heart failure classified as stage B, C or D according to the ACC/AHA classification, and/or wherein the subject having a cardiovascular disease has heart failure according to class Il to IV of the NYHA classification.
19. The method of any of the preceding items, wherein the sodium-glucose cotransporter inhibitor (SGLTi) comprises a sodium-glucose cotransporter 2 inhibitor (SGLT2i) or a sodium-glucose cotransporter 1 inhibitor (SGLT1i), preferably a SGLT2i, more preferably a SGLT2i, more preferably a gliflozin, more preferably comprises canagliflozin, dapagliflozin, empagliflozin, ertugliflozin, ipragliflozin, remogliflozin, sergliflozin, sotagliflozin or tofogliflozin.
20. The method of any of the preceding items, wherein the subject is a human.
21. The method of any of the preceding items, wherein the sample is a blood, serum, plasma or urine sample, preferably the sample is a blood, serum or plasma sample.
22. A computer-implemented method for carrying out the method of any of the preceding items, comprising a) means for determining the amount of FABP3 according to any of items 1-21, or the amount of FABP3 and the cardiac injury marker according to any of items 8-21, in a sample of a subject treated with SGLTi, wherein the subject suffers from a cardiovascular disease; and b) means for comparing said amount of FABP3 to a reference amount according to any of items 1-21, said amount of FABP3 and said amount of the cardiac injury marker to a reference amount according to any of items 8-21, or means for forming and comparing a ratio between said amount of FABP3 and said amount of the cardiac injury marker to a reference ratio according to any of items 9-21.
23. A computer program product comprising a program code for executing the computer-implemented method according to item 22 when run on at least one computer.
24. A device for carrying out the method of any of the preceding items, comprising a) means for determining the amount of FABP3 according to any of items 1-21, or the amount of FABP3 and the cardiac injury marker according to any of items 8-21, in a sample of a subject treated with SGLTi, wherein the subject suffers from a cardiovascular disease; and b) means for comparing said amount of FABP3 to a reference amount according to any of items 1-21, said amount of FABP3 and said amount of the cardiac injury marker to a reference amount according to any of items 8-21, or means for forming and comparing a ratio between said amount of FABP3 and said amount of the cardiac injury marker to a reference ratio according to any of items 9-21.
25. A kit for carrying out the method of any of the preceding items, comprising a) means for determining the amount of FABP3 according to any of claims 1-21, or the amount of FABP3 and the cardiac marker according to any of claims 8-21, in a sample of a subject treated with SGLTi, wherein the subject suffers from a cardiovascular disease; and b) means for comparing said amount of FABP3 to a reference amount according to any of items 1-21, said amount of FABP3 and said amount of the cardiac marker to a reference amount according to any of items 8-21, or means for forming and comparing a ratio between said amount of FABP3 and said amount of the cardiac marker to a reference ratio according to any of items 9-21.
26. Use of i) FABP3 according to any of items 1-21, FABP3 and the cardiac injury marker according to any of items 8-21, and/or ii) use of a detection agent for FABP3, or for FABP3 and the cardiac marker, for detecting the effect induced by a sodium-glucose cotransporter inhibitor (SGLTi) in a subject suffering from a cardiovascular disease.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention. The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group. Further embodiments of the invention will become apparent from the following claims.

1. A method of detecting the effect induced by a sodium-glucose cotransporter inhibitor (SGLTi) in a subject suffering from a cardiovascular disease, the method comprising a) determining the amount of Fatty Acid Binding Protein 3 (FABP3) in a sample of the subject treated with SGLTi;
b) comparing the amount of said FABP3 to a reference amount.

2. The method of claim 1, wherein an increased amount of FABP3 compared to the reference amount is indicative for the effect.

3. The method of claim 1, wherein the reference amount is the amount in a sample of a subject suffering from a cardiovascular disease, or wherein the reference amount is the upper limit of normal (ULN) of the amount in samples of subjects suffering from a cardiovascular disease.

4. The method of claim 1, wherein the reference amount is the amount in a sample of a subject suffering from a cardiovascular disease or the ULN of the amount in samples of subjects suffering from a cardiovascular disease before being treated with SGLTi.

5. The method of claim 1, wherein the method further comprises:
c) determining the amount of FABP3 and a cardiac injury marker in a sample of the subject treated with SGLTi and comparing the amount of said FABP3 and the amount of said cardiac injury marker to the reference amount.

6. The method of any of claim 5, wherein the method further comprises:
d) forming a ratio between the amount of FABP3 and the amount of the cardiac injury marker and comparing the ratio to a reference ratio, wherein an increased ratio between the amount of FABP3 and the amount of the cardiac injury marker compared to the reference ratio is indicative for the effect.

7. The method of claim 1, wherein the amount of FABP3 is monitored for at least 1 hour, at least 1 day, at least 1 week, at least 2 weeks, or at least 4 weeks.

8. The method of claim 5, wherein the cardiac injury marker comprises a cardiac function marker, wherein the cardiac function marker is a natriuretic peptide, wherein the natriuretic peptide comprises Brain Natriuretic Peptide (BNP)-type peptides, and wherein the BNP-type peptides comprise BNP or NT-proBNP.

9. The method of claim 3, wherein the subject having a cardiovascular disease has heart failure, or chronic heart failure.

10. The method of claim 1, wherein the sodium-glucose cotransporter inhibitor (SGLTi) comprises a sodium-glucose cotransporter 2 inhibitor (SGLT2i), a sodium-glucose cotransporter 1 inhibitor (SGLTli), a gliflozin, canagliflozin, a dapagliflozin, an empagliflozin, an ertugliflozin, an ipragliflozin, a remogliflozin, a sergliflozin, a sotagliflozin, or a tofogliflozin.

11. A computer-implemented method for carrying out the method of any claim 1, comprising
a) a means for determining the amount of FABP3 in a sample of a subject treated with SGLTi, wherein the subject suffers from a cardiovascular disease; and
b) a means for comparing said amount of FABP3 to the reference amount, wherein an increased amount of FABP3 compared to the reference amount is indicative for the effect.

12. (canceled)

13. A device for carrying out the method of claim 1, comprising
a) a means for determining the amount of FABP3 in a sample of a subject treated with SGLTi, wherein the subject suffers from a cardiovascular disease; and
b) a means for comparing said amount of FABP3 to the reference amount, wherein an increased amount of FABP3 compared to the reference amount is indicative for the effect.

14. A kit for carrying out the method of claim 1, comprising
a) a means for determining the amount of FABP3 sample of a subject treated with SGLTi, wherein the subject suffers from a cardiovascular disease; and
b) a means for comparing said amount of FABP3 to the reference amount, wherein an increased amount of FABP3 compared to the reference amount is indicative for the effect.

15. (canceled)

16. The method of claim 5, wherein the amount of FABP3 and the cardiac injury marker is monitored for at least 1 hour, at least 1 day, at least 1 week, at least 2 weeks, or at least 4 weeks.

17. A computer-implemented method for carrying out the method of claim 5, comprising
a) a means for determining the amount of FABP3 and the cardiac injury marker in a sample of a subject treated with SGLTi, wherein the subject suffers from a cardiovascular disease; and
b) a means for comparing said amount of FABP3 and said amount of the cardiac injury marker to a reference amount, or means for forming and comparing a ratio between said amount of FABP3 and said amount of the cardiac injury marker to a reference ratio, wherein an increased amount of FABP3 compared to the reference amount, an increased amount of the cardiac injury marker compared to the reference amount, or an increased ratio compared to the reference ratio is indicative for the effect.

18. The computer-implemented method of claim 17, wherein the reference amount is the amount in a sample of a subject suffering from a cardiovascular disease, the amount in a sample from the same subject suffering from a cardiovascular disease, the upper limit of normal (ULN) of the amount in samples of subjects suffering from a cardiovascular disease, the amount in a sample of a subject suffering from a cardiovascular disease before being treated with SGLTi, or the ULN of the amount in samples of subjects suffering from a cardiovascular disease before being treated with SGLTi.

19. A device for carrying out the method of claim 5, comprising
a) a means for determining the amount of FABP3 and the cardiac injury marker in a sample of a subject treated with SGLTi, wherein the subject suffers from a cardiovascular disease; and
b) a means for comparing said amount of FABP3 and said amount of the cardiac injury marker to the reference amount, or means for forming and comparing a ratio between said amount of FABP3 and said amount of the cardiac injury marker to a reference ratio, wherein an increased amount of FABP3 compared to the reference amount, an increased amount of the cardiac injury marker compared to the reference amount, or an increased ratio compared to the reference ratio is indicative for the effect.

20. The device of claim 19, wherein the reference amount is the amount in a sample of a subject suffering from a cardiovascular disease, the amount in a sample from the same subject suffering from a cardiovascular disease, the upper limit of normal (ULN) of the amount in samples of subjects suffering from a cardiovascular disease, the amount in a sample of a subject suffering from a cardiovascular disease before being treated with SGLTi, or the ULN of the amount in samples of subjects suffering from a cardiovascular disease before being treated with SGLTi.

21. A kit for carrying out the method of claim 5, comprising
a) a means for determining the amount of FABP3 and the cardiac injury marker in a sample of a subject treated with SGLTi, wherein the subject suffers from a cardiovascular disease; and
b) a means for comparing said amount of FABP3 and said amount of the cardiac injury marker to the reference amount, or means for forming and comparing a ratio between said amount of FABP3 and said amount of the cardiac injury marker to a reference ratio, wherein an increased amount of FABP3 compared to the reference amount, an increased amount of the cardiac injury marker compared to the reference amount, or an increased ratio compared to the reference ratio is indicative for the effect.

22. The kit of claim 21, wherein the reference amount is the amount in a sample of a subject suffering from a cardiovascular disease, the amount in a sample from the same subject suffering from a cardiovascular disease, the upper limit of normal (ULN) of the amount in samples of subjects suffering from a cardiovascular disease, the amount in a sample of a subject suffering from a cardiovascular disease before being treated with SGLTi, or the ULN of the amount in samples of subjects suffering from a cardiovascular disease before being treated with SGLTi.
